(19) United States
(12) Patent Application Publication
(10) Pub. No.: US 2009/0131790 A1
(43) Pub. Date: May 21, 2009

(54) SYSTEMS AND METHODS FOR DEPLOYING ECHOGENIC COMPONENTS IN ULTRASONIC IMAGING FIELDS

(75) Inventors: Michael A. Munrow, Belmont, CA (US); Craig Gerbi, Mountain View, CA (US); Robert K. Deckman, San Bruno, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Gynesonics, Inc., Redwood City, CA (US)

(21) Appl. No.: 12/119,147

(22) Filed: May 12, 2008

Related U.S. Application Data
(60) Provisional application No. 60/938,140, filed on May 15, 2007.

(51) Int. Cl. A61B 8/00; A61B 18/18; A61M 5/32

(52) U.S. Cl. 600/439; 606/41; 604/272; 604/506

(57) ABSTRACT

Systems and devices according to the present invention providing a needle deployment and visualization device, which includes: a shaft; an ultrasound imaging transducer extendable along at least a portion of the shaft for providing an image within a field of view; and a needle coupled and deployable from the shaft within the field of view. The needle has an artifact configured to preferentially reflect at least a portion of the ultrasound energy emanating from the ultrasound transducer back to the transducer in order to enhance imaging of the needle.

### CROSS-REFERENCES TO RELATED APPLICATIONS

The present application claims the benefit of provisional U.S. Application No. 60/938,140 (Attorney Docket No. 025676-001700US), filed May 15, 2007, the full disclosure of which is incorporated herein by reference.

The present application is also related to but does not claim the benefit of U.S. application Ser. No. 11/620,594 (Attorney Docket No. 025676-000310US), filed Jan. 1, 2007, entitled “Interventional Deployment and Imaging System Grossman;” U.S. application Ser. No. 11/347,018 (Attorney Docket No. 025676-000210US), filed Feb. 2, 2006, entitled “Method and Device for Uterine Fibroid Ablation Grossman;” U.S. patent application Ser. No. 11/409,496 (Attorney Docket No. 025676-000700US) filed Apr. 20, 2006, entitled “Rigid Delivery Systems Having Inclined Ultrasound and Curved Needle;” and U.S. patent application Ser. No. 11/564,164 (Attorney Docket No. 025676-000710US) filed Nov. 28, 2006, entitled “Devices and Methods for Treatment of Tissue,” the disclosures of which are incorporated herein by reference, in their entirety.

### BACKGROUND OF THE INVENTION

The present invention relates generally to medical systems and methods. More particularly, the invention relates to echogenic needles and other components for enhanced visualization of the needles while in use under the guidance of ultrasound imaging techniques.

Current medical treatments of organs and tissues within a patient's body often use a needle or other elongate body for delivery of energy, therapeutic agents or the like. Optionally the methods use ultrasound imaging to observe and identify a treatment target and the position of the needle relative to the treatment target. Such visualization may be difficult, however, particularly if the needle has turned away from the field of view.

Of particular interest to the present invention, a treatment for uterine fibroids has recently been proposed which relies on the transvaginal positioning of a treatment device in the patient's uterus. A radiofrequency or other energy delivery needle is deployed from the device into the fibroid, and energy is delivered in order to ablate the fibroid. To facilitate locating the fibroids and positioning the needles within the fibroids, the device includes an ultrasonic imaging array with a field of view in a generally lateral direction from an axial shaft. A curved needle is advanced from the shaft and into the field of view so that a distal tip of the needle turns away from the transducer as it is being advanced. The geometry of the needle deployment is advantageous since it permits the location and treatment of fibroids which are laterally adjacent to the shaft. The advantages are limited somewhat, however, because it is difficult to accurately image and track the distal tip of the needle as it turns in a direction perpendicular to the transducer which is generally parallel to the field of view.

For these reasons, it would be desirable to provide improved systems and methods for the deployment of needles and other elongate components within ultrasonic fields of view in therapeutic and diagnostic medical devices. It would be particularly desirable if the needles or other elongate components could be modified in some way which would enhance their echogenicity so that the needles, and particularly the distal portions of the needles, would remain more distinctly visible as they are advanced into the field of view and turn away from the ultrasonic transducer or transducer array. It would be further desirable for the modifications to the needle or other elongate device to have little or no adverse or deleterious effects on the performance of the component, to add little or no additional cost to the component, and to add minimum complication to the device fabrication process. At least some of these objectives will be met by the inventions described below.

### BRIEF SUMMARY OF THE INVENTION

The present invention provides both apparatus and methods for visualizing and treating target tissue regions in a patient. The apparatus comprises a shaft or other support which carries an ultrasound imaging transducer or transducer array (referred to herein as “transducer”), typically at or near its distal end. The ultrasound imaging transducer provides imaging within a field of view, typically extending laterally from an axis of the shaft. An elongate component is deployable from the shaft within the field of view so that the component may be imaged as it is advanced through the field of view. In accordance with the principles of the present invention, the component will be modified to have an artifact thereon which is configured to preferentially reflect at least a portion of the ultrasound energy emanating from the ultrasound transducer back to the transducer. In this way, the image of the needle will be enhanced so that it is easier to follow the progress of the needle as it is deployed from the shaft, typically into the tissue being treated.

Usually, the elongate component will comprise a needle, such as an electrically conductive needle for radiofrequency or other therapies. In other instances, however, the needle could be hollow and configured to deliver a therapeutic or other substance into the target tissue. Other elongate components which may be incorporated into the apparatus of the present invention include microwave antennas, cryogenically cooled elements, resistively heated elements, morrelating elements, and the like.

The needle or other elongate component will may be straight, curved, or a combination of straight and curved sections. In the illustrated embodiments, the needle is shown to have a curved or otherwise deflected distal end, where the body of the needle is elastic so that the needle may be retracted into a tubular lumen or other constraint prior to deployment. Such curved elongate components will curve laterally away from the axis of the shaft or other support as they are advanced from the lumen or other restraint. In the case of straight needles, the body may be less elastic or rigid since the straight body can be advanced and retracted in a linear lumen without deformation. With curved needles and other elongate components. The ultrasonic transducer will usually be disposed distally on the tubular lumen or other constraint so that the component enters the field of view in a direction which is initially parallel to the axis and then moves into a direction which is generally perpendicular or transverse to the axis. As the distal end, and in particular the distal tip, of the elongate component moves into a generally transverse or perpendicular orientation, it will be appreciated that the effective cross-sectional area decreases and the visibility of the distal end diminishes. By providing artifacts which preferentially reflect ultrasonic energy back toward the transducer, however, the image of the distal end can be significantly enhanced. A variety of particular artifacts will be described in more detail below.

The artifact may reflect the ultrasonic energy back to the transducer in a wide variety of ways. The ultrasound energy preferably is reflected back from the artifact to the transducer along substantially the same line. In some embodiments, the artifact may comprise retroreflector, such as a corner retroreflector having at least two and sometimes three mutually perpendicular surfaces forming substantially 90 degrees angles. In other embodiments the artifact may be located near or at a needle distal tip. The needle distal tip may comprises an asymmetric or offset trocar tip. In still other embodiments the artifact is in the form of a needle distal tip including a series of steps providing surfaces parallel, the steps decreasing widths as the steps approach the needle distal tip. In yet other embodiments, the needle distal tip includes a plurality of beveled surfaces which offset a variety of angles and a plurality beveled surfaces providing parallel surfaces In further exemplary embodiments, the needle distal tip includes a plurality of indentations to aid in reflection of the ultrasound energy. The indentions may be oriented in parallel to one another, may be oriented perpendicularly to one another, or may be oriented at other relative angles. Alternatively, the artifacts may comprise one or more indentations extending longitudinally along a long axis of the needle distal tip. In some embodiments, the at least one indentation substantially has a circular or oval geometry extending axially and/or may circumferentially at a location on or proximal to the needle distal end. Other indentations may be centered relative to the long axis of the needle distal tip.

In an embodiment, the echogenic needle which is configured for the delivery of the RF energy is inserted within the patient's body, prior, concurrent, or after the insertion of the ultrasound probe insert. In an embodiment, and as used herein after, for purposes of describing the inventive features of the present invention, and not by way of limitation, the ultrasound probe is an insertable probe and includes an array located, preferably, at its distal end for delivering ultrasound energy to the needle.

Often, the actual tip (e.g., from which RF energy is delivered to the target site) of the needle is not directly viewable on the imaging screen, thus making visualization and navigation of the needle within the uterine difficult. By way of example, the needle may be positioned within the body such that its most distal tip is not within the field of view or may scatter the ultrasound waves which have been directed to it and not reflect the waves back to the array. In some instances it may be shadowed by the rest of the ultrasound array, such that the sound waves emanating from the array and onto the needle are not necessarily reflected back to the array, or at least not reflected back from the most distal tip of the needle which would normally be delivering the RF energy for the ablation of the fibroid.

In an embodiment, in order to improve the visualization of the needle under ultrasound, one or more artifacts are incorporated in the needle at a known distance from its most distal tip. In an embodiment, since the one or more artifacts are at least relatively well identifiable by the reflection of the ultrasound waves back to the array, and are located at a known distance from the needle distal tip (or at the distal tip), the visualization system and/or the user can determine the location of the needle distal tip, even if the distal tip is not directly visible.

In an embodiment, the artifact may be either or both a retroreflector and a reflective surface which is at least substantially parallel to the ultrasound array, when located within the patient's body in the deployed position. In either case, the one or more artifacts enable reflecting of at least a portion of the ultrasound waves along the same line back to the array. Given the known features of the artifact and its distance from and/or relationship to needle distal tip, the position of the distal tip of the needle may be determined, even in the absence of direct view of the needle distal tip on the imaging screen.

In an embodiment, the artifact is a retroreflector. As used herein, retroreflector refers to a device, artifact, or feature which sends light, or other sources of radiation including ultrasound waves back to where they emanated from regardless of the angle of incidence.

In an embodiment, the retroreflector is of a corner cube type. A corner cube retroreflector provides a three mutually perpendicular surfaces which form a corner, thus called a corner reflector or corner cube. In an embodiment, the corner is at least substantially 90 degrees, preferably 90 degrees. The angle of the reflected ultrasound energy from the artifact may range from −0.25 radians to about 0.25 radians.

In an embodiment, the artifact is a retroreflector located at a pre-determined distance from the needle distal tip. In an embodiment, the retroreflector is of a corner cube type. In an embodiment, the corner cube retroreflector is in the form of a cutout section in the body of the needle and is located at a pre-determined distance from the needle distal tip.

In some embodiments, the artifact may be partially retroreflective by creating or depositing a layer of tiny refractive spheres on the surface (e.g., of the needle) or by creating small pyramid like structures (e.g., behaving in essence as a corner reflector). In both cases the internal reflection causes the waves to be reflected back to where it originated.

In an embodiment, the reflective surface of the artifact is at least substantially parallel to the array when the needle is at least partially deployed, preferably, when the needle is at full deployment at the desired location.

In an embodiment, the longitudinal dimension of the reflective surface of the artifact ranges from about one to about eight times that of the wavelength of the ultrasound energy, generally, from about 1 to about 8 times, normally being at least substantially equal to the wavelength. The longitudinal dimension of the array, usually ranges from about 2 to about 24 mm, normally about 12 mm.

Although in further describing the invention, solid tip will be used as the needle distal tip, it should be appreciated that the same principles may also be applicable to hollow tips. The solid tip may take on a variety of geometries in accordance with the intended use. In some embodiments, the tip may comprise any one or more of the following shapes, configurations, and shapes: an asymmetric or offset trocar tip, a plurality of beveled edges offset at a variety of angles, a barbed tip having corners, one or more indentations, or combinations thereof. In some embodiments, the one or more indentations may be one or a plurality of transverse cuts in the tip; one or a plurality of longitudinal cuts in the tip; one or a plurality of circular or oval shaped indentations or dimples (similar to a golf ball) in the distal tip. In an embodiment, the distal tip may be attached, by way of example, laser welded, to the needle hollow body and including a plurality of transverse cuts extending circumferentially around an area proximal to the distal tip. The circumferential cuts may be transversely set apart at pre-determined distance from one another.

In an embodiment of the present invention, a delivery system, preferably rigid delivery system, includes a rigid delivery shaft, an imaging core, and an interventional core. The rigid delivery shaft will have a proximal end, a distal end, and an axial passage. The axial passage will typically extend the entire length of the shaft from the proximal to distal end and be open at least at the proximal end. The shaft will usually be rigid along all or a portion of its length, but in other instances may be flexible, deflectable, or steerable.

In an embodiment, a device includes a rigid delivery system, preferably a rigid delivery system, having an ultrasound array for improved imaging and curved needle for ablation treatment. The ultrasound array may have a tilt relative to an axis of a flexible shaft of the ultrasound insert or may be parallel to the ultrasound shaft axis in the relaxed state (e.g., prior to insertion in the rigid delivery system). The system allows for needle deployment into solid tissue under direct, usually real-time, visualization and/or ultrasound visualization. Typically, the needle having one or more artifacts/features as described above, will be deployed from within a natural or created body cavity or body lumen. Exemplary body cavities include the uterus, the esophagus, the stomach, the bladder, the colon, and the like. Exemplary body lumens include the ureter, the urethra, fallopian tubes, and the like. Created body cavities include insufflated regions in the abdomen, the thoracic cavity, regions around joints (for arthroscopic procedures), and the like. The present invention will generally not find use with procedures in blood vessels or other regions of the vasculature. Thus, while the following description will be directed particularly at procedures within the uterus for detecting and treating uterine fibroids, the scope of the present invention is not intended to be so limited.

The imaging core preferably comprises an ultrasound imaging insert or probe disposed within the axial passage, usually being removably disposed so that it may be removed and replaced to permit sterilization and re-use. The imaging insert will have an ultrasound array within a distal portion thereof. The ultrasound array may or may not be tilted relative to an ultrasound shaft axis. In an embodiment, the array runs parallel to the ultrasound shaft axis when in the relaxed configuration and prior to insertion into the rigid shaft of the delivery system. In an embodiment, the ultrasound array may be tilted at an angle in a range from about 7 degrees to about 15 degrees, preferably in a range from about 7 degrees to about 10 degrees. It will be appreciated that the interventional core may be adapted for any conventional form of medical imaging, such as optical coherence tomographic imaging, direct optic visualization, and as such is not limited by ultrasonic imaging. Furthermore, as indicated above, it should be appreciated that the echogenic needles as described in relation for use with treatment of a patient using radio frequency energy, are also usable in application of other forms of treatment, including but not limited to drug delivery, biopsy, and the delivery of other forms of energy and the like. It should be appreciated by those skilled in the art, that as indicated above, the imaging core may be an external probe.

In an embodiment, the ultrasound imaging insert further comprises a flat viewing window disposed over the ultrasound array at the distal portion. The distal end of the rigid shaft may comprise a mechanical alignment feature, as for example, a flat viewing surface for axial or rotational orientation of the ultrasound imaging insert within the shaft. The flat viewing surface will be visually transparent to permit imaging from within the axial passage by the imaging insert. It will be appreciated, however, that the transparent visualization surface which aids in physical alignment does not have to be visually transparent for ultrasound. For example, at least a portion of the flat viewing surface may be composed of an ultrasonically translucent material to permit ultrasonic imaging through the surface of the shaft. Further, the re-usable ultrasound imaging insert may be acoustically coupled to the outer delivery shaft to ensure that the ultrasound energy effectively passes from one component to the other. Ultrasonic acoustic coupling may be accomplished in several ways by one or a combination of means, including a compliant material (e.g., pad, sheet, etc.), fluid (e.g., water, oil, etc.), gel, or close mechanical contact between the rigid shaft and ultrasound imaging insert. In some embodiments, the ultrasound array has a length of 12 mm and a depth of 3 mm.

In an embodiment, the rigid delivery shaft preferably has a deflectable or fixed pre-shaped or pre-angled distal end. The delivery shaft distal end may be deflected or bent at an angle in a range from about 0 degrees to about 90 degrees relative to the shaft axis, preferably in a range from about 10 degrees to about 25 degrees. The ultrasound imaging insert will usually be flexible (and in some instances deflectable or steerable) so that the distal portion of the ultrasound imaging insert is conformable or bendable to the same angle as the shaft deflectable distal end. In an embodiment, the viewing angle of the ultrasound imaging insert is zero (i.e., angle due to tilted ultrasound array in the relaxed state prior to insertion into the rigid shaft passage), and the viewing angle is equal to the shaft bending relative to the rigid shaft axis.

In some embodiments, when the array of the ultrasound imaging insert in the relaxed state prior to insertion into the rigid shaft has a tilt relative to the ultrasound imaging insert axis, the cumulative effect of array tilting and shaft bending may advantageously provide an enhanced viewing angle of the ultrasound imaging insert. The viewing angle may range from about 0 (when the array is not tilted in the relaxed state). In some embodiments, when the array is tilted relative to the ultrasound shaft axis in the relaxed state, the viewing angle may range from about 7 degrees (i.e., angle due to tilted ultrasound array) to about 90 degrees relative to the shaft axis. In one embodiment, the viewing angle is about 20 degrees, wherein the array tilting and shaft bending are at about 10 degrees respectively, or when the array tilting is 0 and the shaft bending is 20 degrees. It will be appreciated that several geometries of array tilting and shaft bending may be configured so as to provide the desired viewing angle (e.g., distally forward direction, side-viewing or lateral direction), as for example, viewing of the end within the uterus (e.g., cornua and fundus).

In an embodiment, the interventional core preferably comprises a curved echogenic needle coupled to the rigid shaft via a needle guide. Significantly, an angle of needle curvature is dependent upon (e.g., inversely proportional to) the ultrasound array tilt (when the array is tilted and has an angle relative to the ultrasound shaft axis) and the rigid shaft bent. For example, an increase in an angle of array tilting or shaft bending decreases the degree of needle curvature needed to stay within the filed of view of the imaging transducer. This in turn provides several significant advantages such as allowing a treating physician or medical facility to selectively choose an appropriate needle curvature based upon such indications (e.g., variability in needle curvature). Further, a decrease in the angle of needle curvature provides for enhanced pushability, deployability, and/or penetrability characteristics as well as simplified manufacturing processes. The angle of needle curvature may be in a range from about 0 degrees to about 80 degrees relative to a needle axis which runs parallel to the proximal un-curved portion of the needle. Preferably the angle is about 70 degrees when the viewing angle is about 20 degrees. The curved needle generally comprises a two-piece construction comprising an elongate hollow body and a solid or hollow distal tip, and an artifact/feature as described above.

In an embodiment, the echogenic needle extends adjacent an exterior surface of the rigid delivery shaft. In an embodiment, the needle is disposed within a needle guide which extends along an exterior of the rigid shaft. The echogenic needle may be removably and replaceably disposed within the guide passage. The guide passage will typically extend approximately the entire length of the shaft and be open at least at the distal end so as to allow the needle to be reciprocatably deployed and penetrated into adjacent solid tissue. In an embodiment, the needle has a hollow body and a solid distal tip formed from conductive material.

In an embodiment, the curved needle and needle guide have a flattened oval shape that has a wideness that is greater than a thickness. This oval cross sectional shape is intended to inhibit lateral deflection during deployment or penetration of the needle. The echogenic needle is configured to deliver to the target site radio frequency energy (or other ablative energy such as, but not limited to, electromagnetic energy including microwave, resistive heating, cryogenic). In some embodiments, the energy, such as the RF energy, is generated at a relatively low power and for relatively a short duration of active treatment time.

In an embodiment, a delivery system includes a shaft, an imaging core, and an interventional core. The delivery shaft has a proximal end, an angled distal tip, and an axial passage therethrough. The imaging core comprises an ultrasound imaging insert disposed within the axial passage. The imaging insert has an ultrasound array within a distal portion thereof. The ultrasound array may or may not be tilted relative to an ultrasound shaft axis when the insert is in a free configuration (e.g., before insertion into the rigid shaft). The interventional core comprises a curved ablation needle coupled to the shaft. An angle of needle curvature may be inversely proportional to the ultrasound array tilt, if the array is tilted, and tip angle.

As discussed above, the geometries of the shaft, imaging insert, treatment needle, needle distal tip, and needle guide may be varied in accordance with the intended use. The delivery shaft, ultrasound imaging insert, treatment needle, and/or needle guide may be integrally formed or fixed with respect to one another or preferably comprise separate, interchangeable modular components that are coupleable to one another to permit selective sterilization or re-use, and to permit the system to be configured individually for patients having different anatomies and needs. For example, a sterilizable and re-usable ultrasound insert may be removably positioned within a disposable shaft.

The target site undergoing treatment may be any target site which may benefit from the treatment devices and methods according to the present invention. Usually the target site is a uterus within a female's body. The target site in need of treatment generally has an initial (e.g., prior to treatment) approximate diameter which is greater than about two (2) centimeters (“cm”). Usually, the target site's initial diameter ranges from about 1 to about 6 cm. Normally the initial untreated diameter is about 2 cm.

In an embodiment of methods according to the present invention for visualization of an ultrasound reflective object within a patient's body, include inserting a needle to a target site within the patient's body, with the needle being configured for use with a device for the treatment of the target site. The needle has an artifact/feature located thereon, preferably distal end, which is configured to reflect ultrasound energy which is deliverable to the artifact, back to the ultrasound energy source at an angle of reflection which is substantially equal to an angle of incidence of the ultrasound energy. In an embodiment, the ultrasound energy is delivered to the artifact by way of an ultrasound array located, preferably, at a distal end of an ultrasound insert. In an embodiment, the ultrasound insert and the needle are inserted and delivered to the target site with the ultrasound energy delivered to the artifact on the needle. The artifact may be in the form of a retroreflector or a reflective surface which is parallel to the ultrasound array. The method may further include, establishing the position of the distal tip of the needle based on the reflection of the ultrasound energy back to the array and its known distance from the needle distal tip, and the viewing plane observable on the ultrasound system.

In an embodiment of methods according to the present invention for visualization and ablation of fibroid tissues needing treatment within a patient's body include providing a visualization and ablation system according to the device and system embodiments described herein. In an embodiment, the method comprises inserting a rigid shaft having a proximal end, a distal end, and an axial passage therethrough within a uterus. The distal end of the rigid shaft may then be selectively deflected. An ultrasound imaging insert may then be loaded within the axial passage prior to, concurrent with, or subsequent to shaft insertion. In some embodiments, a distal portion of the insert conforms to the deflected shaft distal end. Loading may further involve axially or rotationally aligning the ultrasound imaging insert within the rigid shaft. A needle curvature is then selected by the physician or medical facility from a plurality of needles (i.e., at least two or more) having different curvatures based on at least an angle of the deflected shaft distal end. The selected curved needle is then loaded along the rigid shaft. Under the guidance of the imaging system, the needle is inserted into the tissue site. The RF generator is set to deliver and/or maintain a target temperature at the target site for a treatment period. The needle has an artifact/feature located thereon, preferably at the distal end, which is configured to reflect ultrasound energy which is deliverable to the artifact, back to the ultrasound energy source at an angle of reflection which is substantially equal to an angle of incidence of the ultrasound energy.

In an embodiment, the ultrasound array may be tilted or inclined within the distal portion of the insert, wherein selecting the needle curvature further comprises accounting for the ultrasound array tilt. In some embodiments, the ultrasound array may run parallel to the axis of the ultrasound insert itself. As described above, if the ultrasound array is tilted, it is preferably tilted at an angle in a range from about 7 degrees to about 10 degrees relative to an ultrasound shaft axis. The inclination will usually be fixed, but in some instances the distal tip may be actively deflectable. Deflecting will typically comprise pulling a pull or tensioning wire coupled to the shaft distal end in a proximal direction. Deflection occurs at an angle in a range from about 0 degrees to about 80 degrees relative to the shaft axis, wherein the needle curvature is in a range from about 0 degrees to about 90 degrees (i.e., in the case of a non-tilted ultrasound array) relative to an axis. The method further comprises imaging the uterus with a viewing angle of the ultrasound array in a range from about 0 degrees to about 90 degrees (i.e., in the case of a straight needle) relative to the shaft axis, wherein the viewing angle is based upon the deflected shaft distal end and the ultrasound array. It will be appreciated that torquing and/or rotating the rigid device in addition to tip deflection and ultrasound tilt will allow a physician to obtain the desired viewing plane.

In some embodiments, methods further include ablating a uterine fibroid within the uterus with the selected curved needle. In those cases, the needle may be a radiofrequency (RF) electrode, a microwave antenna, a cryogenic probe, or other energy delivery or mediating element intended for ablating or otherwise treating tissue. The distal tip of the needle will usually be adapted so that it will self-penetrate into the tissue as it is advanced from the needle guide. The direction of advancement will be coordinated with the imaging field of the ultrasound insert so that the penetration of the curved needle can be viewed by the physician, usually in real time. Further, an electrolyte (e.g., saline) or other agent may be infused within the uterus prior to or concurrently with fibroid ablation so as to enhance the therapeutic effect provided by the treatment needle. This is preferably accomplished by providing at least one or more (e.g., two, three, four, five, etc.) infusion holes or apertures on the needle body. In still other cases, the needle could be a hollow core needle intended for sampling, biopsy, otherwise performing a diagnostic procedure.

A further understanding of the nature and advantages of the present invention will become apparent by reference to the remaining portions of the specification and drawings.

### BRIEF DESCRIPTION OF THE DRAWINGS

The following drawings should be read with reference to the detailed description. Like numbers in different drawings refer to like elements. The drawings, which are not necessarily to scale, illustratively depict embodiments of the present invention and are not intended to limit the scope of the invention.

FIGS. 1A though 1D illustrate an exemplary delivery system having an ultrasound array for imaging and an echogenic needle for improved imaging and ablation treatment embodying features of the present invention.

FIG. 2A is a schematic illustration of a corner cube retroreflector according to the present invention.

FIGS. 2B and 2C are schematic illustrations comparing the behavior of parallel and non-parallel surfaces in reflecting sound waves back to the source.

FIG. 3 illustrates of the system of FIG. 1A within a uterus for the treatment of fibroids in accordance with the principles of the present invention.

FIGS. 4A-4E illustrate an exemplary asymmetric solid distal tip embodying features of the present invention.

FIGS. 5A-5J illustrate distal portions of various echogenic needles embodying features of the present invention.

FIGS. 6A and 6B illustrate an echogenic needle having a transponder at a distal portion.

### DETAILED DESCRIPTION OF THE INVENTION

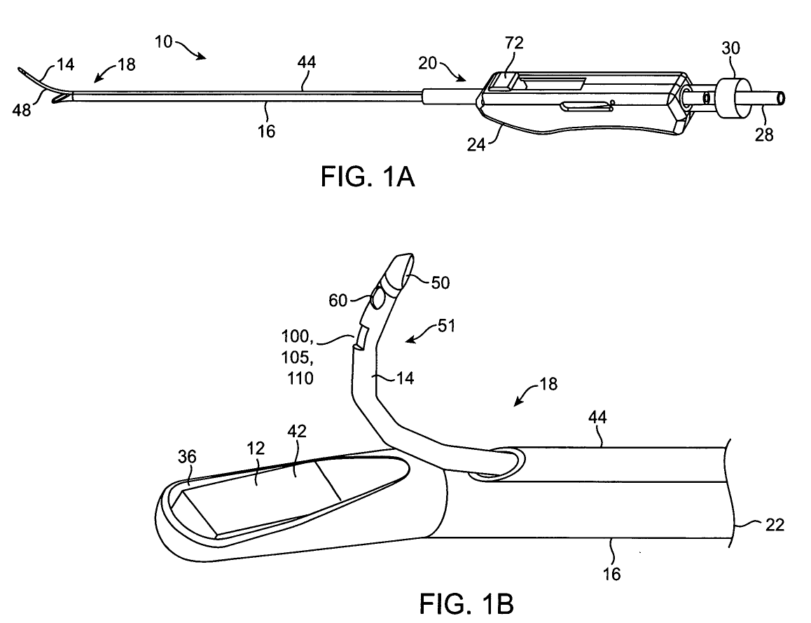

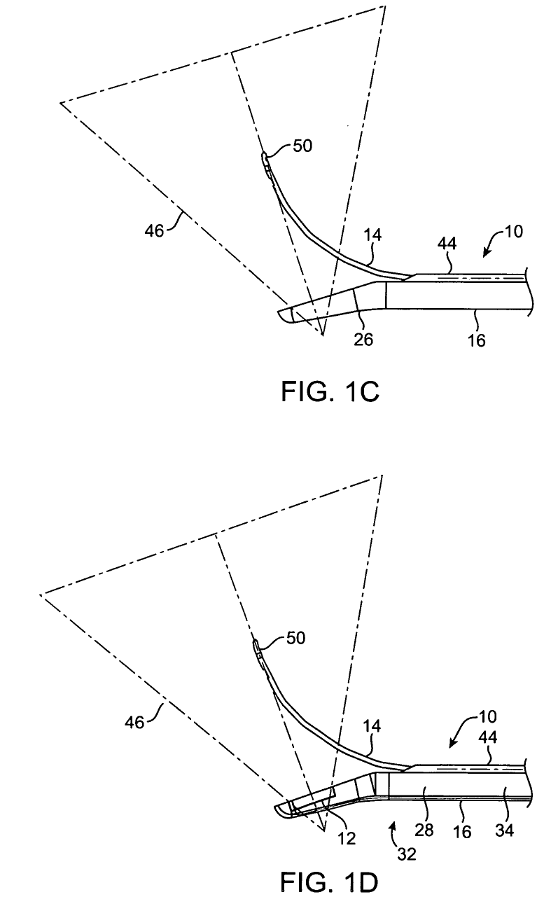

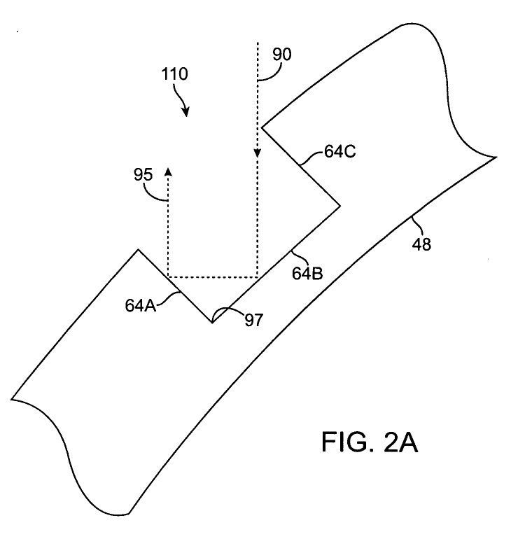

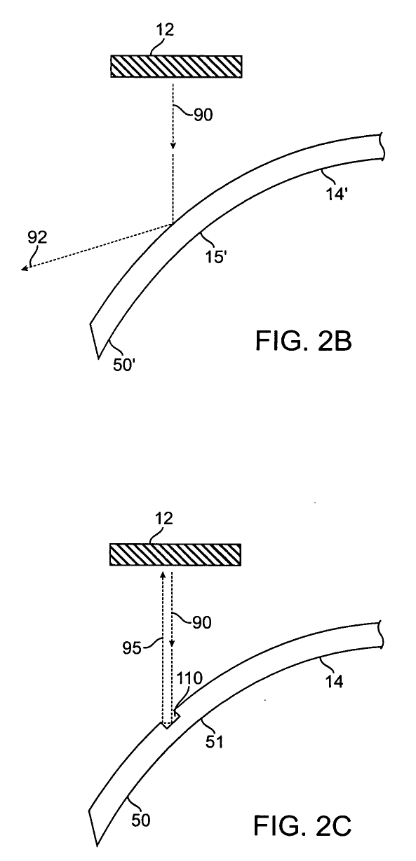

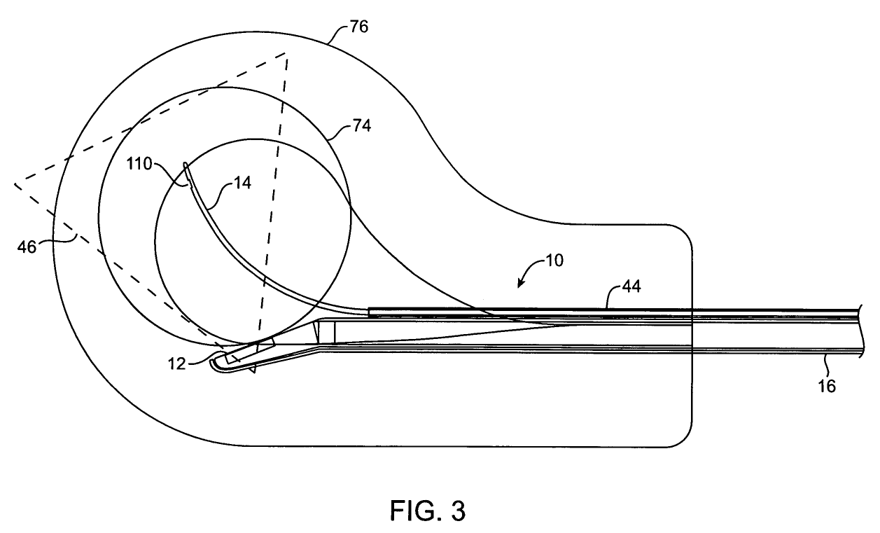

Referring now to FIGS. 1A through 1D, an exemplary deflectable tip delivery system 10 having an ultrasound array 12 (inclined as shown) for improved imaging and curved needle 14 for ablation treatment of a target site such as fibroid tissues 74 (FIG. 3) within a female's reproductive system, such as uterus 76, is illustrated. The system 10 generally includes a rigid or other delivery shaft 16, an ultrasound imaging insert 28, and an echogenic curved needle 14 with an artifact/feature 100 at a distal end 51 thereof. As shown, the artifact is a retroreflector 105 of a corner cube type 110. The delivery shaft 16 comprises a distal end 18, a proximal end 20, and an axial passage 22 for housing the ultrasound imaging insert 28 therein. A handle 24 may be attachable to the proximal end 20 of the shaft 16. The distal end 18 of the shaft 16 may have a bent or deflectable distal tip 26, as best seen in FIGS. 1B and 1C. The ultrasound imaging insert 28 includes a flexible shaft 34 (see FIG. 1D), the ultrasound array 12 at a distal portion 32 thereof, and a flat viewing window 36 within the distal portion 32 (see FIG. 1D). A corner cube reflector is more clearly shown in FIG. 2A. below.

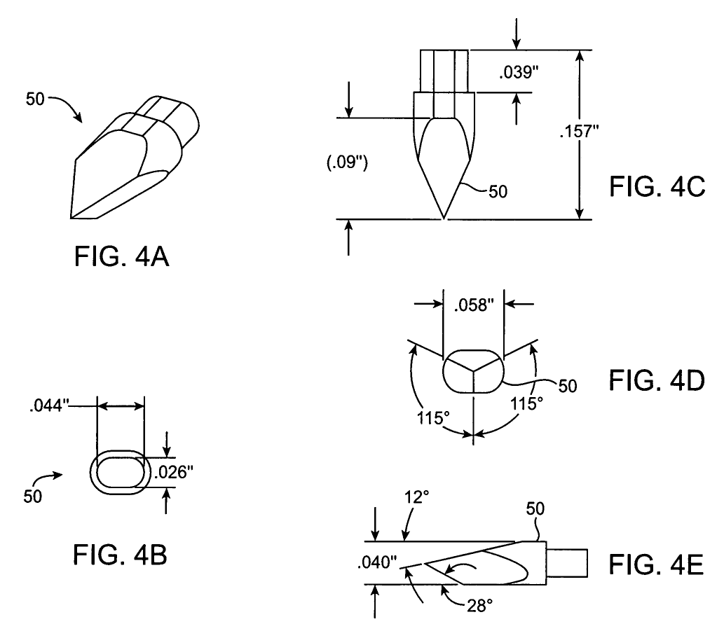

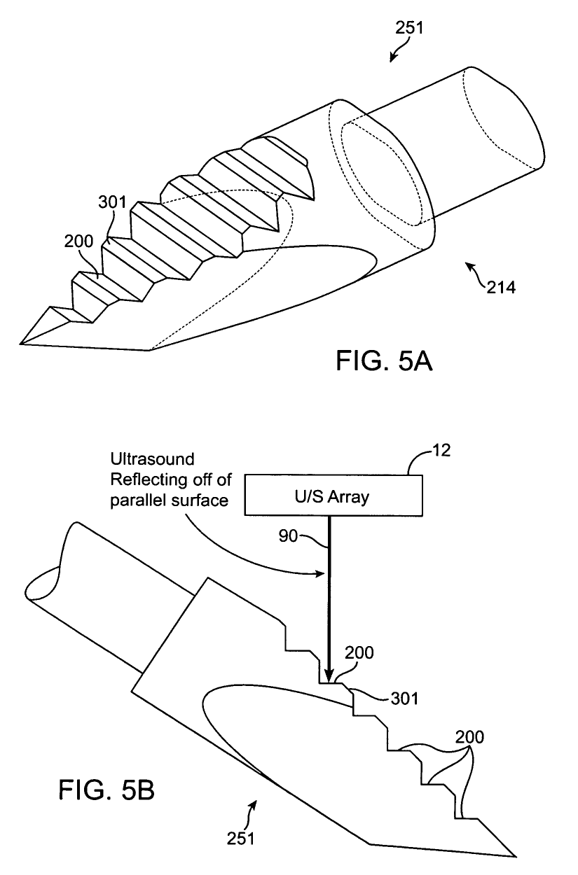

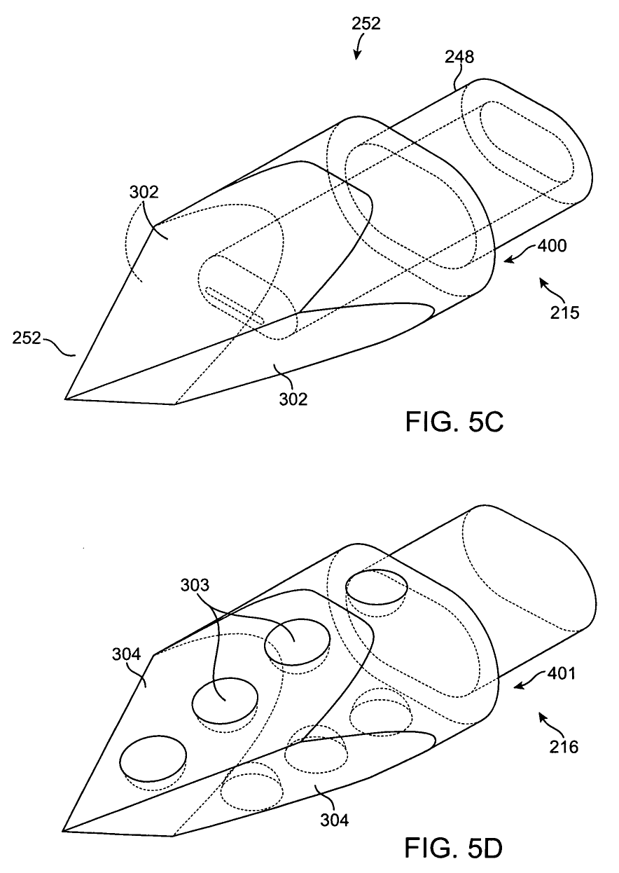

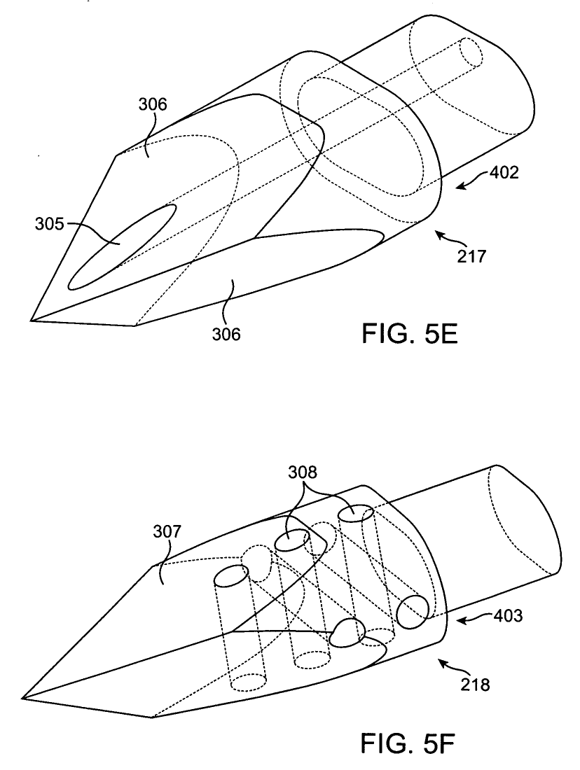

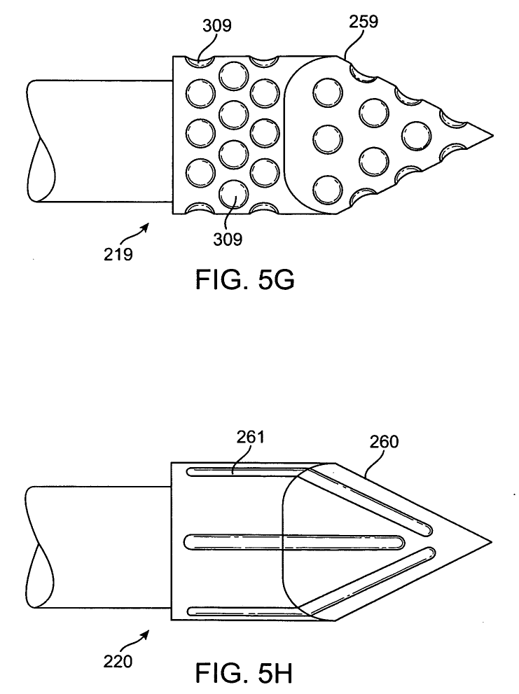

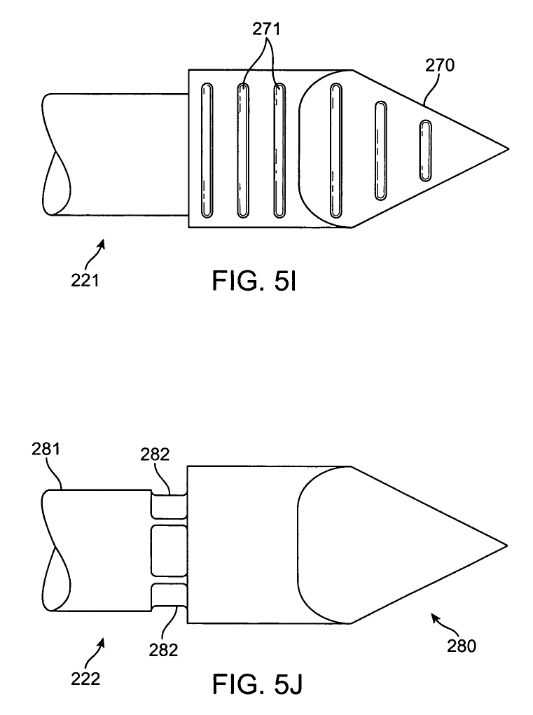

The needle 14 is shown as a solid tip electrically conductive needle intended for radiofrequency tissue ablation. As discussed elsewhere, it could also be a hollow core needle intended for substance delivery or injection. The exemplary needle 14 generally comprises a two-piece construction comprising an elongate hollow body 48 (as best seen in FIG. 4A) and a solid distal tip 50 at a distal end 51 thereof. The distal tip 50 may be laser welded to the hollow tubular body 48. The solid tip 50 may also be attached via alternative means, for example adhesives or mechanical features or fits. The hollow tube 48 will generally have a length in a range from about 20 cm to about 45 cm. In some embodiments, the hollow tube will have an oval cross section having a thickness generally in a range from about 0.5 mm to about 2 mm and a wideness generally in a range from about 1 mm to about 3 mm. This flattened oval cross sectional shape, when present, and as shown in FIG. 5B is intended to inhibit lateral deflection during deployment or penetration of the needle 14. FIG. 1B also illustrates a representative laser cut hole 60 within the distal end 51 of the tubular body 48 for the infusion of agents (e.g., electrolytes, drugs, etc.) so as to enhance the therapeutic effect of the needle 14 prior to or during ablation treatment. The infusion hole 60 may be aligned on one side of the tubular body 48 and generally has length in a range from about 0.5 mm to about 2 mm and a width in a range from about 0.5 mm to about 2 mm. It should be noted that hole 60 may comprise one or a plurality of holes, and each may be used for a different purpose.

The handle 24 further includes a longitudinally movable slider 72 for enabling the advancement and retraction of the needle 14 to and from within a needle guide 44. The ultrasound imaging insert 28 may be removably and replaceably disposed within the axial passage 22 of the shaft 16. A sealing element 30 may be provided between the ultrasound imaging insert 28 and the shaft handle 24 to ensure sufficient sealing around the insert 28 at a proximal end. It will be appreciated that the above depictions are for illustrative purposes only and do not necessarily reflect the actual shape, size, or dimensions of the system 10. Furthermore, the ultrasound array may be parallel to an axis of the ultrasound array. This applies to all depictions hereinafter. The array is typically a linear array with from 16 to 128 elements, usually having 64 elements. The length (azimuth) of array 12 usually ranges from about 5 mm to about 20 mm, normally being about 14 mm. The array may have a depth (elevation) ranging from about 1 mm to about 4 mm, normally being about 2 mm. In an embodiment, the ultrasound array transmits ultrasound waves at a center frequency ranging from about 2 MHz to about 15 MHz, typically from about 5 MHz to about 12 MHz, normally about 6.5 MHz.

Now referring to FIG. 2A, a corner cube reflector is more clearly illustrated. As can be seen, the corner cube retroreflector 110 has three mutually orthogonal reflecting surfaces 64A, 64B, and 64C. The corner cube reflects the ultrasound waves back towards their source, here the array 12. The reflection angle at 180° is independent of the orientation of the corner cube, thus the reflected waves 95 are generally parallel to the incident waves 90.

Now referring to FIGS. 2B and 2C, simplified illustrations of an array and a needle with and without an echogenic imparting artifactor feature incorporated therein are shown to further illustrate the advantages of the present invention. As can be seen from FIG. 2A, in the absence of the artifact, the ultrasound waves 90 emanated from the array 12 and directed toward the needle 14′ (without the artifact) are scattered 92 and do not return to the array. Now referring to FIG. 2B, the needle 14 includes the corner cube artifact 110 at its distal end 51 near the distal tip 50. In the exemplary embodiment shown in FIG. 2B, the incidence waves 90 are directed toward the needle 14, and are reflected 95 in a substantially parallel geometry as related to the incidence waves back to the array 12. In the FIG. 2B as shown, the ultrasound waves emanating from the array 12 hit the needle 14 at the artifact 110. In this embodiment, since the corner cube retroreflector is placed at a pre-determined distance from the needle distal tip, the position of the distal tip can be more accurately determined.

Referring to FIG. 3, use of the system 10 for treating fibroids 74 in a uterus 76 will be described. The shaft 16 is advanced so that the transducer 12 is located within the interior of the uterus to locate fibroid 74 within a field of view 46. The needle 14 may then be axially advanced from the needle/guide 44 so that its distal portion curves or deflects laterally outwardly within the field of view 46 and into the fibroid 74. As the needle advances, the distal end turns away from the surface of the transducer 12 until it eventually becomes perpendicularly aligned, as shown in FIG. 3. In this perpendicular alignment, the artifact 110 will continue to preferentially direct ultrasound signals back to the transducer in order to enhance the image of the distal end of the needle within the ultrasonic view.

Now referring to FIG. 4A-4E, the solid tip 50 may comprise an asymmetric or offset trocar tip. The center point of the tip 50 may be offset from a centerline of the needle to help compensate for any needle deflections due to tenacious tissue, in effect steering the needle towards the intended target even with the deflection. For example, the tip 50 may comprise a plurality of beveled edges offset at a variety of angles as illustrated in FIGS. 4D and 4E. It will be appreciated that the solid tip 50 may comprise a variety of dimensions and shapes and is not limited to FIGS. 4A, 4B, 4C, 4D, and 4E. It will be further appreciated that the tip 50 need not be a separate component but may alternatively be integrally formed with the needle body 48. The needle 14, including the tip 50 and tubular body 48 may be formed from a variety of materials including stainless steel, nitinol, and like materials for transmitting ablation energy.

Now referring to FIGS. 5A to 5J, various embodiments of artifacts which may provide a reflective surface are illustrated.

FIGS. 5A and 5B illustrate a needle 214 having a distal tip portion 251. The distal tip 251 includes a plurality of steps 301. The steps 301, as best shown in FIG. 5B, provide surfaces 200 which are oriented to preferentially reflect ultrasound energy back to the array.

FIG. 5C, illustrates a distal portion of a needle 215, having a trocar tip 252 with a barbed region 400 disposed about a distal end of a needle body 248. The distal tip and the body 248 may be laser welded together or formed as a single integral piece. The needle distal tip includes three beveled edges 302, offset at angles to provide enhanced reflective surfaces. FIG. 5D illustrates a distal portion of a needle 216, similar to the needle 215 of FIG. 5C, further having a plurality of circular indentations 303, on one or more beveled surfaces 304 thereof.

FIG. 5E illustrates a distal portion of a needle 217, similar to the needle 215 of FIG. 5C, further having an oval aperture 305, on one or more beveled surfaces 306 for providing reflective surfaces. FIG. 5F illustrates a distal portion of a needle 218 having a plurality of cylindrical apertures 308 on various surfaces 307 of the distal tip for providing reflective surfaces.

FIG. 5G illustrates a distal portion of a needle 219. A distal tip 259 includes a plurality of indentations 309, similar to dimples on a golf ball for providing reflective surfaces. FIG. 5H illustrates a distal portion of a needle 220 having a distal tip 260 which includes a plurality of parallel longitudinal cuts 261, along the surface of the tip. The cuts 261 provide additional reflective surfaces.

Similarly FIG. 5 illustrates a distal tip 270 at a distal portion of needle 221 including a plurality of transverse cuts 271, over the surface of the tip for providing reflective surfaces. FIG. 5J illustrates a distal portion of a needle 222 having a distal tip 280 attached to a needle body 281. The needle body 281 includes a plurality of transverse cuts 282 immediately proximal to the distal tip which provide additional reflective surfaces.

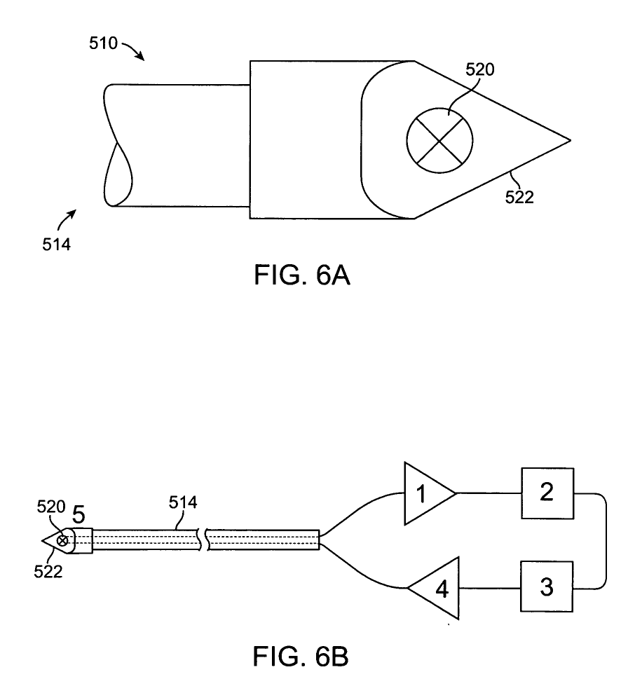

Now referring to FIGS. 6A and 6B, a distal portion 510 of a needle 514 is illustrated. A transponder 520 is located at a distal tip 522 of the needle. The transponder receives the ultrasound waves in a receiver 1, processes the signal in processors 2 and 3, and a transmitter 4 transmits a signal back to the ultrasound system for displaying image and/or position information on an imaging screen.

Although certain exemplary embodiments and methods have been described in some detail, for clarity of understanding and by way of example, it will be apparent from the foregoing disclosure to those skilled in the art that variations, modifications, changes, and adaptations of such embodiments and methods may be made without departing from the true spirit and scope of the invention. Therefore, the above description should not be taken as limiting the scope of the invention which is defined by the appended claims.

What is claimed is:

1. A visualization and treatment device, comprising:
a support;
an ultrasound imaging transducer on the shaft to provide imaging within a field of view; and
an elongate component deployable from the shaft within the field of view;
wherein the component has an artifact configured to preferentially reflect at least a portion of the ultrasound energy emanating from the ultrasound transducer back to the transducer.

2. The device of claim 1, wherein the artifact is located near or at a distal tip of the elongate component.

3. The device of claim 2, wherein the ultrasound energy is reflected from the artifact back to the transducer substantially along the same line.

4. The device of claim 3, wherein the artifact comprises a retroreflector formed on or in the component.

5. The device of claim 4, wherein the retroreflector comprises at least two mutually perpendicular surfaces forming substantially 90 degrees angles.

6. The system of claim 3, wherein the artifact comprises a series of steps on the shaft parallel surfaces with widths which decrease as they approach the distal tip.

7. The system of claim 3, wherein the artifact comprises a plurality of beveled surfaces offset a variety of angles, the beveled surfaces providing parallel surfaces.

8. The system of claim 3, wherein the artifact comprises a plurality of indentations to aid in reflection of the ultrasound energy.

9. The system of claim 8, wherein the indentions have surfaces at different angles relative to one another.

10. The system of claim 8, wherein the indentions have surfaces parallel angles relative to one another.

11. The system of claim 8, wherein the indentions have surfaces at perpendicular angles relative to one another.

12. The system of claim 8, wherein the at least one indentation extends longitudinally along a long axis of the needle distal tip.

13. The system of claim 8, wherein the at least one indentation substantially has a circular or oval geometry.

14. The system of claim 8, wherein the at least one indentation includes one or more indentations extending circumferentially at a location proximal the needle distal end.

15. The system of claim 8, wherein the indentations are aligned along a long axis of the needle distal tip.

16. The system of claim 8, wherein the indentations are offset from the long axis of the needle distal tip.

17. The system of claim 8, wherein the circumferential indentations are circumferentially set apart at a pre-determined distance from one another.

18. The system of claim 1, wherein the artifact comprises a transponder located at the distal end of the elongate component for receiving the ultrasound energy and transmitting information to provide the location of the needle distal tip.

19. The system of claim 1, wherein the elongate component comprises a needle.

20. The system of claim 19, wherein the needle is a solid tip needle configured to deliver electrical energy into tissue.

21. The system of claim 19, wherein the needle is a hollow needle configured to deliver a substance into tissue.

22. The system of claim 19, wherein the needle has a trocar tip.

23. The system of claim 22, wherein the trocar tip is asymmetric or offset.

24. A method for deploying an elongate component under direct visualization, said method comprising:
positioning a shaft in a body cavity;
visualizing a target region in the body cavity within a field of view using an ultrasonic transducer on the shaft; and
advancing the elongate component from the shaft toward the target region within the field of view;
wherein ultrasonic energy is preferentially reflected back to the transducer by an artifact on the elongate member to enhance production of an image of the elongate member.

25. A method as in claim 24, wherein the elongate component is advanced so that a distal tip of the elongate member moves into a parallel orientation relative to the field of view.

26. A method as in claim 25, wherein the artifact includes at least one surface which moves into a transverse orientation relative to the field of view as the elongate component is advanced, wherein the at least one surface reflects energy from the transducer back to the transducer.

27. A method as in claim 25, wherein the elongate member is a needle advanced into tissue.

28. A method as in claim 27, further comprising delivering energy into the tissue.

29. A method as in claim 26, wherein the tissue is uterine tissue and the needle is advanced into or adjacent to a fibroid.

30. A method as in claim 27, further comprising injecting a substance into the tissue through the needle.
